(19) United States
(12) Patent Application Publication
(10) Pub. No.: US 2021/0127761 A1
(43) Pub. Date: May 6, 2021

(54) SINGLE-PIECE GARMENT

(71) Applicant: Perla Andrea Murray, Fort Lauderdale, FL (US)

(72) Inventor: Perla Andrea Murray, Fort Lauderdale, FL (US)

(21) Appl. No.: 17/086,784

(22) Filed: Nov. 2, 2020

Related U.S. Application Data
(60) Provisional application No. 62/928,871, filed on Oct. 31, 2019.

(51) Int. Cl. A41D 11/00; A41B 9/08; A61F 13/496

(52) U.S. Cl. CPC A41D 11/00; A41B 9/08; A61F 2013/422; A41D 2300/322; A61F 13/496

(57) ABSTRACT

Disclosed herein is a garment in accordance with some embodiments. Accordingly, the garment comprises a front panel, a rear panel, and a fastener. Further, the rear panel is attached to the front panel along a periphery of the rear panel in at least a part forming a torso portion and two leg portions of the garment. Further, a lower torso portion of the torso portion and an upper leg portion of each leg portion of the two leg portions forms a crotch portion. Further, the crotch portion comprises a slit. Further, the slit is configured to provide access to at least the part of the torso and the leg through the slit. Further, the fastener is disposed on the crotch portion. Further, a second fastener element of the fastener and a first fastener element of the fastener is configured to be detachably fastened for openably closing the slit.

The current application claims a priority to the U.S. Provisional Patent application Ser. No. 62/928,871 filed on Oct. 31, 2019. The current application is filed on Nov. 2, 2020 while Oct. 31, 2019 was on a weekend.

### FIELD OF THE INVENTION

Generally, the present disclosure relates to a field of apparel. More specifically, the present disclosure relates to a single-piece garment.

### BACKGROUND OF THE INVENTION

Rompers for babies of sizes ranging from 0-3 months to 2T is a convenient single piece garment that can be worn during any time of the day, as well as an easy pick for parents and caregivers while packing dress for babies. Some rompers in the market don't have any access to the diaper area, in which case you have to get the baby completely undressed for changing their diaper. Some others have an opening with a fastener in between the legs. The majority of such rompers have snap buttons in the diaper area which can be quite frustrating to put together when you are changing the diaper. The functionality of being able to zip the romper in one easy swoop is incredibly helpful for parents and/or caregivers while changing the baby's diaper. Further, most zippered rompers in the market do not have a protective fabric underneath to prevent any pinching. Furthermore, most zippered rompers in the market have their zipper handles exposed, which can lead to the baby playing with it and/or unzipping the romper.

Therefore, there is a need for an improved single-piece garment that may overcome one or more of the above-mentioned problems and/or limitations.

### SUMMARY OF THE INVENTION

This summary is provided to introduce a selection of concepts in a simplified form, that are further described below in the Detailed Description. This summary is not intended to identify key features or essential features of the claimed subject matter. Nor is this summary intended to be used to limit the claimed subject matter's scope.

Disclosed herein is a garment in accordance with some embodiments. Accordingly, the garment may include a front panel, a rear panel, and at least one fastener. Further, the rear panel may be attached to the front panel along a periphery of the rear panel in at least a part forming a torso portion of the garment and two leg portions of the garment. Further, the torso portion may include a neck opening disposed on an upper torso end of the torso portion. Further, the torso portion may include two arm openings disposed on two opposing torso side ends of the torso portion. Further, each leg portion of the two leg portions may include a leg opening disposed on a lower leg end of the each leg portion. Further, the torso portion may be configured to accommodate at least a part of a torso of an individual. Further, the each leg portion may be configured to accommodate at least a part of a leg of the individual. Further, a lower torso portion of the torso portion and an upper leg portion of the each leg portion may form a crotch portion. Further, the crotch portion may include a slit. Further, the slit may include a first end and a second end. Further, the slit may be configured to provide access to a crotch of the individual through the slit. Further, the at least one fastener may be disposed on the crotch portion. Further, the at least one fastener may include a first fastener element and a second fastener element. Further, the first fastener element may be disposed on a first crotch portion of the crotch portion corresponding to a first side of the slit. Further, the second fastener element may be disposed on a second crotch portion of the crotch portion corresponding to a second side of the slit. Further, the second fastener element and the first fastener element may be configured to be detachably fastened for openably closing the slit. Further, the slit may transition between an open state and a closed state based on the openably closing. Further, the crotch may be accessible through the slit in the open state. Further, the crotch may not be accessible through the slit in the closed state.

Further disclosed herein is a garment in accordance with some embodiments. Accordingly, the garment may include a front panel, a rear panel, and at least one fastener. Further, the rear panel may be attached to the front panel along a periphery of the rear panel in at least a part forming a torso portion of the garment and two leg portions of the garment. Further, the torso portion may include a neck opening disposed on an upper torso end of the torso portion. Further, the torso portion may include two arm openings disposed on two opposing torso side ends of the torso portion. Further, each leg portion of the two leg portions may include a leg opening disposed on a lower leg end of the each leg portion. Further, the torso portion may be configured to accommodate at least a part of a torso of an individual. Further, the each leg portion may be configured to accommodate at least a part of a leg of the individual. Further, a lower torso portion of the torso portion and an upper leg portion of the each leg portion may form a crotch portion. Further, the crotch portion may include a slit. Further, the slit may include a first end and a second end. Further, the each leg portion may include an outer leg portion edge defining the leg opening. Further, at least one of the first end and the second end may be discontiguous with the outer leg portion edge of the each leg portion. Further, the slit may be configured to provide access to a crotch of the individual through the slit. Further, the at least one fastener may be disposed on the crotch portion. Further, the at least one fastener may include a first fastener element and a second fastener element. Further, the first fastener element may be disposed on a first crotch portion of the crotch portion corresponding to a first side of the slit. Further, the second fastener element may be disposed on a second crotch portion of the crotch portion corresponding to a second side of the slit. Further, the second fastener element and the first fastener element may be configured to be detachably fastened for openably closing the slit. Further, the slit may transition between an open state and a closed state based on the openably closing. Further, the crotch may be accessible through the slit in the open state. Further, the crotch may not be accessible through the slit in the closed state.

Both the foregoing summary and the following detailed description provide examples and are explanatory only. Accordingly, the foregoing summary and the following detailed description should not be considered to be restrictive. Further, features or variations may be provided in addition to those set forth herein. For example, embodiments may be directed to various feature combinations and sub-combinations described in the detailed description.

### BRIEF DESCRIPTION OF THE DRAWINGS

The accompanying drawings, which are incorporated in and constitute a part of this disclosure, illustrate various embodiments of the present disclosure. The drawings contain representations of various trademarks and copyrights owned by the Applicants. In addition, the drawings may contain other marks owned by third parties and are being used for illustrative purposes only. All rights to various trademarks and copyrights represented herein, except those belonging to their respective owners, are vested in and the property of the applicants. The applicants retain and reserve all rights in their trademarks and copyrights included herein, and grant permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure.

FIG. 1 is a front view of a garment, in accordance with some embodiments.

FIG. 2 is a rear view of the garment, in accordance with some embodiments.

FIG. 3 is a partial front perspective view of a crotch portion of a garment, in accordance with some embodiments.

FIG. 4 is a front perspective view of the crotch portion, in accordance with some embodiments.

FIG. 5 is a partial front perspective view of the crotch portion, in accordance with some embodiments.

FIG. 6 is a partial front perspective view of the crotch portion, in accordance with some embodiments.

FIG. 7 is a partial front perspective view of the crotch portion, in accordance with some embodiments.

FIG. 8 is a partial perspective view of a garment, in accordance with some embodiments.

FIG. 9 is a front perspective view of a garment worn by an individual, in accordance with some embodiments.

FIG. 10 is a front perspective view of a garment worn by an individual, in accordance with some embodiments.

FIG. 11 is a table of a bill of material associated with a garment, in accordance with some embodiments.

FIG. 12 is a front view of a garment, in accordance with some embodiments.

### DETAIL DESCRIPTIONS OF THE INVENTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim limitation found herein and/or issuing here from that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present disclosure. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the claims found herein and/or issuing here from. The present disclosure contains headers. It should be understood that these headers are used as references and are not to be construed as limiting upon the subjected matter disclosed under the header.

The present disclosure includes many aspects and features. Moreover, while many aspects and features relate to, and are described in the context of a single-piece garment, embodiments of the present disclosure are not limited to use only in this context.

Overview:

The present disclosure describes a single-piece garment. Further, the single-piece garment may include a romper. Further, the romper may include a zipper romper. Further, the zipper romper may include a concealed zipper in between the legs. The zipper romper comprises a small pocket on one leg of the romper, wherein the small pocket can hide the zipper handle in a concealed and neat fashion. The zipper romper further comprises a protective fabric that runs all along underneath the zipper, thereby preventing any pinching on the baby during zipping or unzipping. Furthermore, the zipper romper comprises a flexible material and stretchable stitching, that enable users to slip the romper on and off of the baby through an opening in the neck area, or through the zipper area in between the legs. Further, the zipper extends from a position slightly above the leg hole of one leg to a similar position on the other leg, which prevents the zipper from getting completely undone. Further, the zipper romper may include a concealed zippered romper that is easy to handle for parents, as well as comfortable to wear for babies and toddlers. Further, the zipper romper has a zipper, whose length extends from the bottom region of one leg to the bottom region of the other leg, along the crotch area of the romper. Further, the zipper can be of any material, features, type or kind, such as metal, nylon coil, molded plastic, separating, double-ended, etc. Further, the zipper romper comprises an invisible kind of zipper with a metal handle. Further, it is preferred that the length of the zipper is long enough, such that the zipper romper enables parents/caregivers to take the legs of a child out of the leg holes through the zipper area, and the lower section of the child can be accessed without the romper coming in the way while diaper changing. The zipper romper can be of any size, material, features, type or kind, orientation, location, quantity, components, and arrangements of components. Further, the romper may be made from a material that is lightweight, stretchable, breathable, durable, machine-washable, easily manufacturable, flexible, non-toxic, environmentally safe, and/or hypoallergenic. Further, the material may include fabrics, polyester, nylon, cotton, spandex, etc. Further, any designs or patterns on a romper with a concealed zipper in between the legs, will fall under the scope of the present disclosure.

Further, the zipper romper comprises a handle pocket for the zipper handle. The handle pocket can be of any shape, size, material, features, type or kind, orientation, location, quantity, components, and arrangements of components. Further, the handle pocket is made of the same material as the romper, and the handle pocket has dimensions big enough to hold, store, and retrieve the handle of the zipper comfortably by the user. Further, the handle pocket is located at the fastening end of the zipper, at a position slightly above the leg hole of one leg of the romper. Further, a small section near the leg hole is left without the zipper on both legs, so that the zipper does not get completely undone. This helps the user to quickly fasten and unfasten the zipper without the fear of completely unfastening the zipper and trying to put the zipper handle back in the zipper track. Further, the zipper romper may include a protective fabric running underneath the complete length of the zipper, such that it prevents any pinching during the operation of the zipper. Further, the protective fabric comprises the same material as the material of the romper. Furthermore, it is preferred that the zipper romper comprises a stretchable stitching technique, that enables the user to put the zipper romper on and off of a baby like a T-shirt, through the neck opening, or through the opening in the diaper area by unzipping the zipper. Further, the invisible zipper and the zipper handle pocket provides a clean and fine hemline in between the legs of the zipper romper, while still providing the ease of handling the garment in different situations for the user. Further, the zipper romper may be specifically made for different seasons with different materials, wherein apart from the zipper in between the legs, additional openings and fasteners may be enabled for ease of wearing the zipper romper.

Further, the present disclosure describes a method of operation of the zipper romper for changing the diaper or removing the zipper romper. Further, the method may include unzipping the zipper and taking the legs of the baby out of the leg hole through the zipper area, to completely access the lower section of the baby without the romper coming in the way. After changing the diaper, the legs of the baby could be put in through the leg holes, the zipper fastened, and the zipper handle secured in the handle pocket.

Further, the present disclosure describes a concealed zippered romper that enables parents to easily put on and take off, of their babies, as well as conveniently change the diaper of their babies without entirely removing the zipper romper.

Further, the present disclosure describes a zipper romper that has a concealed zipper in between the legs. The zipper romper comprises a small pocket on one leg of the romper, wherein the small pocket can hide the zipper handle in a concealed and neat fashion. The zipper romper comprises a protective fabric that runs all along underneath the zipper, thereby preventing any pinching on the baby while zipping or unzipping. Furthermore, the zipper romper comprises a flexible material and stretchy stitching, that enable users to slip the romper on and off of the baby through a neck opening, or the zipper area in between the legs. Further, the zipper extends from a position slightly above the leg hole of one leg to a similar position on the other leg, which prevents the zipper from getting completely undone. Further, the zipper romper may include a concealed zippered romper that is easy to handle for parents as well as comfortable to wear for babies and toddlers.

Further, the present disclosure generally relates to rompers and/or garments More specifically, the present disclosure describes a romper with a concealed zipper in between the legs.

Further, the present disclosure describes a romper with a concealed zipper. Further, the romper may include an inseam. Further, the zipper is disposed on the inseam concealing the zipper. Further, the concealing of the zipper may make the zipper invisible.

Further, the present disclosure describes a sleeveless one piece garment with a zip at a front crotch. Further, the sleeveless one piece garment may include a garment. Further, the sleeveless one piece garment may include a self-fabric binding around a neck with topstitching. Further, the sleeveless one piece garment may include ¼″ double-needle cover-stitching around collar band seam. Further, the sleeveless one piece garment may include ⅜ ″ collar banding around the neck and armholes. Further, the sleeveless one piece garment may include 2 ply folded finish rib cuff. Further, the sleeveless one piece garment may include an invisible zipper with an interior zipper guard at a crotch seam. Further, the sleeveless one piece garment may include light weight fusible interfacing interior.

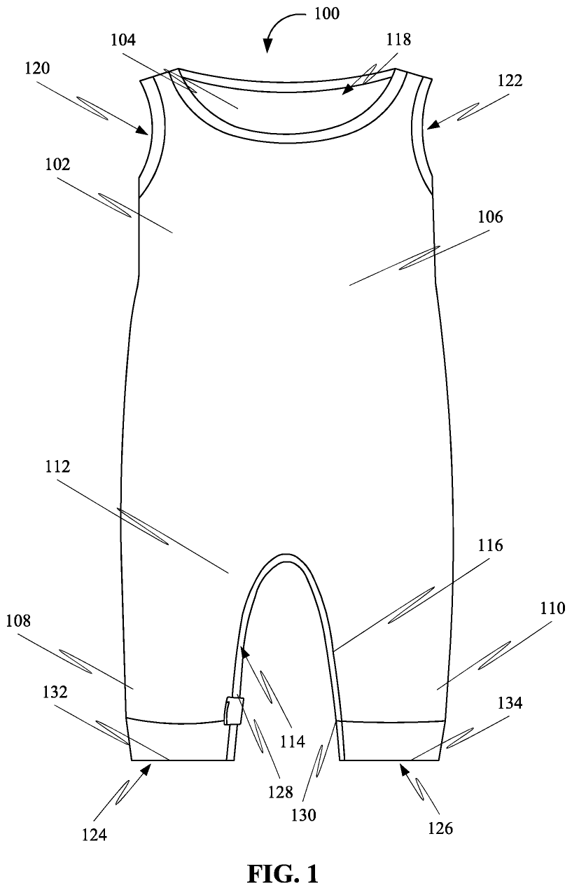

FIG. 1 is a front view of a garment 100, in accordance with some embodiments. Further, the garment 100 may include a front panel 102, a rear panel 104, and at least one fastener 116. Further, the garment 100 may be a single-piece garment. Further, the single-piece garment may include a romper. Further, the romper may include a zipper romper. Further, the zipper romper may include a concealed zipper in between the legs. Further, the garment 100 may be a sleeveless one piece garment with a zipper.

Further, the rear panel 104 may be attached to the front panel 102 along a periphery of the rear panel 104 in at least a part forming a torso portion 106 of the garment 100 and two leg portions 108-110 of the garment 100. Further, the torso portion 106 may include a neck opening 118 disposed on an upper torso end of the torso portion 106. Further, the torso portion 106 may include two arm openings 120-122 disposed on two opposing torso side ends of the torso portion 106. Further, each leg portion of the two leg portions 108-110 may include a leg opening 124-126 disposed on a lower leg end of the each leg portion. Further, the torso portion 106 may be configured to accommodate at least a part of a torso of an individual. Further, the each leg portion may be configured to accommodate at least a part of a leg of the individual. Further, a lower torso portion of the torso portion 106 and an upper leg portion of the each leg portion may form a crotch portion 112. Further, the crotch portion 112 may include a slit 114. Further, the slit 114 may include a first end 128 and a second end 130. Further, the slit 114 may be configured to provide access to a crotch of the individual through the slit 114.

Further, the at least one fastener 116 may be disposed on the crotch portion 112. Further, the at least one fastener 116 may include a first fastener element and a second fastener element. Further, the first fastener element may be disposed on a first crotch portion of the crotch portion 112 corresponding to a first side of the slit 114. Further, the second fastener element may be disposed on a second crotch portion of the crotch portion 112 corresponding to a second side of the slit 114. Further, the second fastener element and the first fastener element may be configured to be detachably fastened for openably closing the slit 114. Further, the slit 114 may transition between an open state and a closed state based on the openably closing. Further, the crotch may be accessible through the slit 114 in the open state. Further, the crotch may not be accessible through the slit 114 in the closed state.

In further embodiments, at least one protective panel may be attached to at least a part of an inner surface of the crotch portion 112. Further, the at least one protective panel may be configured to extend over the inner surface of the crotch portion 112 for covering the slit 114. Further, the at least one protective panel may be configured to prevent interaction between the at least one fastener 116 and the crotch of the individual based on the covering. Further, the interaction may include pinching, touching, etc. Further, the at least one protective panel may include at least one material. Further, the at least one material may include at least one of at least one stretchable material and at least one flexible material. Further, the at least one protective panel may include at least one protective fabric.

Further, in some embodiments, the at least one fastener 116 may include a zipper. Further, the zipper may be disposed on at least one location on the crotch portion 112 making the zipper invisible. Further, the first fastener element may include a first zipper element and the second fastener element may include a second zipper element. Further, the first zipper element and the second zipper element may be a zipper track. Further, the zipper may include a slider operationally coupled with the first zipper element and the second zipper element. Further, the slider may be configurable for moving between a plurality of positions along the first zipper element and the second zipper element for detachably fastening the first zipper element and the second zipper element. Further, in an embodiment, the first zipper element may extend between the first end 128 and the second end 130 on the first crotch portion.

Further, the second zipper element may extend between the first end 128 and the second end 130 on the second crotch portion. Further, the slider may move between the plurality of positions between the first end 128 and the second end 130 based on at least one external action receivable on at least one portion of the slider. Further, the at least one portion of the slider may be a handle. Further, the at least one external action may include a pulling action, a pushing action, etc. Further, the plurality of positions may include an open position and a closed position. Further, the slider may be positioned proximally to the first end 128 and distally from the second end 130 in the open position. Further, the slider may be positioned proximally to the second end 130 and distally from the first end 128 in the closed position. Further, in an embodiment, at least one first panel may be attached to at least a part of an outer surface of the crotch portion 112 along a periphery of the at least one first panel in at least a part forming a receptacle and an opening leading into the receptacle. Further, the at least one first panel may be proximal to at least one of the first end 128 and the second end 130. Further, the receptacle may be configurable for removably receiving the at least one portion of the slider through the opening. Further, receiving of the at least one portion of the slider in the receptacle may prevent the at least one portion of the slider form receiving the at least one external action. Further, the at least one first panel may include at least one material. Further, the at least one material may include at least one of at least one stretchable material and at least one flexible material. Further, the at least one first panel may include at least one fabric.

Further, in some embodiments, the each leg portion may include an outer leg portion edge 132-134 defining the leg opening 124-126. Further, at least one of the first end 128 and the second end 130 may be discontiguous with the outer leg portion edge 132-134 of the each leg portion.

Further, in some embodiments, the each leg portion may include an outer leg portion edge 132-134 defining the leg opening 124-126. Further, at least one of the first end 128 and the second end 130 may be contiguous with the outer leg portion edge 132-134 of the each leg portion.

Further, in some embodiments, the rear panel 104 may be detachably attached to the front panel 102 along the periphery of the rear panel 104 in at least the part forming at least one of the torso portion 106 of the garment 100 and the two leg portions 108-110 of the garment 100 using at least one fastening element. Further, in an embodiment, the at least one fastening element may include a primary fastening element and a secondary fastening element. Further, the primary fastening element may be disposed along a periphery of the front panel 102. Further, the secondary fastening element may be disposed along the periphery of the rear panel 104. Further, the primary fastening element may be configured to be detachably fastened to the secondary fastening element for detachably attaching the rear panel 104 to the front panel 102.

Further, in some embodiments, the rear panel 104 may be fixedly attached to the front panel 102 along the periphery of the rear panel 104 in at least the part forming the torso portion 106 of the garment 100 and the two leg portions 108-110 of the garment 100. Further, the rear panel 104 may be stitched to the front panel 102 along the periphery of the rear panel 104 in at least the part forming the torso portion 106 of the garment 100 and the two leg portions 108-110 of the garment 100 using flexible stitching. Further, the flexible stitching allows stretching of at least one of the neck opening 118, the two arm openings 120-122, the leg opening 124-126, and the slit 114.

Further, in some embodiments, at least one of the front panel 102 and the rear panel 104 may include at least one material. Further, the at least one material may include at least one of at least one stretchable material and at least one flexible material. Further, the at least one material allows at least one of donning and removing of the garment 100 from a body of the individual through at least one of the neck opening 118 and the slit 114. Further, in an embodiment, the at least one material allows the at least one of the donning and the removing of at least one of the two leg portions 108-110 from the leg through the slit 114 without the at least one of the donning and the removing of the torso portion 106 from the torso.

In further embodiments, at least one absorbing layer may be attached to at least a part of an inner surface of the crotch portion 112. Further, the at least one absorbing layer may include at least one absorbing material. Further, the at least one absorbing material may be configured for absorbing a liquid disposable on the at least one absorbing layer.

In further embodiments, at least one cushioned layer may be attached to at least a part of an inner surface of the two leg portions 108-110. Further, the at least one cushioned layer may include at least one cushion material. Further, the at least one cushion material may be configured for providing cushioning to at least the part of the leg.

Further, in some embodiments, at least one of the front panel 102 and the rear panel 104 may include at least one indicator material. Further, the at least one indicator material may be configured for reversibly changing coloring of the at least one indicator material between at least one first color and at least one second color in response to an interaction of the at least one indicator material with at least one substance in at least one quantity. Further, the at least one substance may be produced by the individual. Further, the at least one substance may include at least bodily fluid. Further, the at least one bodily fluid may include urine, sweat, saliva, etc. Further, changing of the coloring from the at least one first color to the at least one second color may indicate a presence of the at least one substance. Further, reversing of the coloring from the at least one second color to the at least one first color may indicate an absence of the at least one substance.

Further, in some embodiments, at least one of the front panel 102 and the rear panel 104 may include at least one first indicator material. Further, the at least one first indicator material may be configured for reversibly changing coloring of the at least one first indicator material between at least one first color and at least one second color based on an application of heat in at least one quantity to the at least one indicator material. Further, the heat may be associated with a temperature of the individual. Further, changing of the coloring from the at least one first color to the at least one second color may indicate an increase in the temperature from at least one first value of the temperature to at least one second value of the temperature. Further, reversing of the coloring from the at least one second color to the at least one first color may indicate a decrease in the temperature from the at least one second value to the at least one first value.

Further, in some embodiments, at least one of the front panel 102 and the rear panel 104 may include at least one second indicator material. Further, the at least one second indicator material may be configured for reversibly changing coloring of the at least one second indicator material between at least one first color and at least one second color based on an application of force in at least one quantity to the at least one second material. Further, the force may include a tension force applied to the at least one second material based on a change in a weight of a diaper worn by the individual within the garment 100. Further, changing of the coloring from the at least one first color to the at least one second color may indicate an increase in the force from at least one first value of the force to at least one second value of the force. Further, reversing of the coloring from the at least one second color to the at least one first color may indicate a decrease in the force from the at least one second value to the at least one first value.

Further, in some embodiments, the torso portion 106 may include two sleeve portions. Further, a proximal end of each sleeve portion of the two sleeve portions may be attached to each arm opening of the two arm openings 120-122. Further, the each sleeve portion may include a sleeve opening disposed on a distal end of the each sleeve portion. Further, the each sleeve portion may be configured to accommodate an arm of the individual.

In further embodiments, at least one cover panel may be configured for covering at least a part of an outer surface of the crotch portion 112. Further, the at least one cover panel may cover at least one of the at least one fastener 116 and the slit 114 based on the covering. Further, the at least one cover panel may include a fixed periphery and a free periphery. Further, the at least one cover panel may be attached to the outer surface along the fixed periphery. Further, the at least one cover panel may be transitionable between a cover state and a revealed state. Further, the at least one cover panel juxtaposes at least the part of the outer surface in the cover state concealing the at least one of the at least one fastener 116 and the slit 114. Further, the concealing of the at least one fastener 116 may make the at least one fastener 116 invisible. Further, the at least one cover panel displaces away from at least the part of the outer surface in the revealed state revealing the at least one of the at least one fastener 116 and the slit 114. Further, the revealing of the at least one fastener 116 may make the at least one fastener 116 visible. Further, the at least one cover panel may include at least one material. Further, the at least one material may include at least one of at least one stretchable material and at least one flexible material. Further, the at least one cover panel may include at least one fabric.

Further, in some embodiments, the crotch portion 112 may include at least a part of at least one inseam. Further, the at least one inseam may include attaching in at least a part forming the slit 114 on the at least one inseam. Further, the at least one fastener 116 may be disposed on the at least one inseam. Further, the at least one fastener 116 may include a zipper.

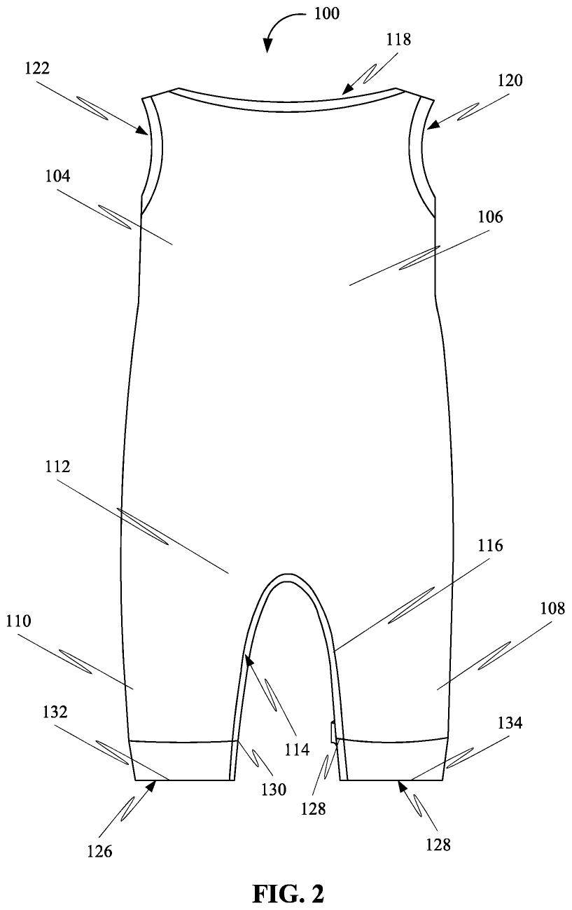

FIG. 2 is a rear view of the garment 100, in accordance with some embodiments.

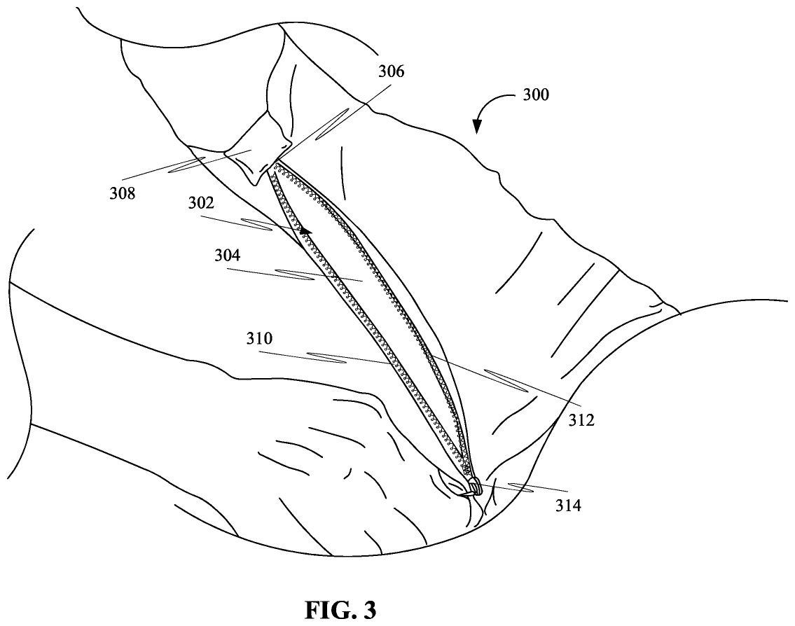

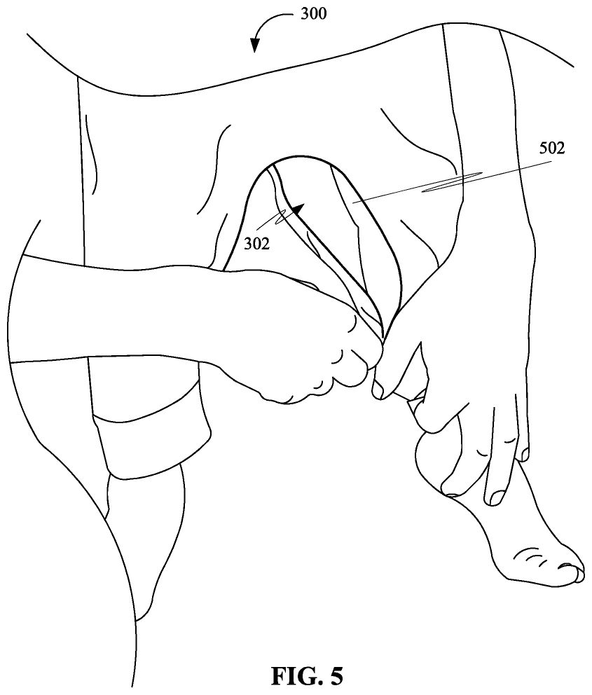

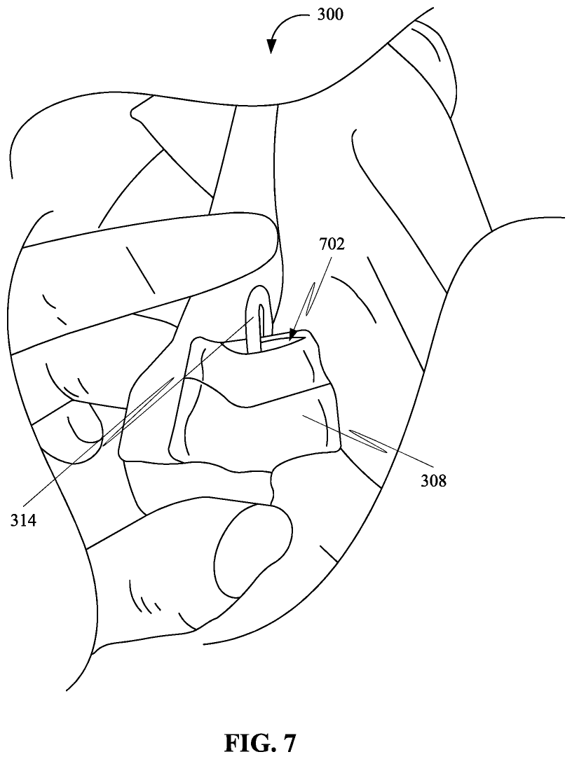

FIG. 3 is a partial front perspective view of a crotch portion 300 of a garment, in accordance with some embodiments. Further, the crotch portion 300 may include a slit 302. Further, the slit 302 may include a first end 306 and a second end (not shown). Further, the slit 302 may be configured to provide access to a crotch 502, as shown in FIG. 5, of an individual through the slit 302. Further, the crotch 502 may be a diaper area. Further, the garment may include a zipper disposed on the crotch portion 300. Further, the zipper may include a first zipper element 310, a second zipper element 312, and a slider 314. Further, the first zipper element 310 may include a zipper track. Further, the second zipper element 312 may be the zipper track. Further, the slider 314 may be operationally coupled with the first zipper element 310 and the second zipper element 312. Further, the slider 314 may be configurable for moving between a plurality of positions along the first zipper element 310 and the second zipper element 312 for detachably fastening the first zipper element 310 and the second zipper element 312. Further, the first zipper element 310 may extend between the first end 306 and the second end. Further, the second zipper element 312 may extend between the first end 306 and the second end. Further, the slider 314 may move between the plurality of positions between the first end 306 and the second end based on at least one external action receivable on at least one portion of the slider 314. Further, the at least one portion of the slider 314 may include a handle. Further, the plurality of positions may include an open position and a closed position. Further, the garment may include at least one first panel 308 attached to at least a part of an outer surface of the crotch portion 300 along a periphery of the at least one first panel 308 in at least a part forming a receptacle 702, as shown in FIG. 7, and an opening leading into the receptacle 702. Further, the at least one first panel 308 may be proximal to at least one of the first end 306 and the second end. Further, the receptacle 702 may be configurable for removably receiving the at least one portion of the slider 314 through the opening. Further, receiving of the at least one portion of the slider 314 in the receptacle 702 may prevent the at least one portion of the slider 314 form receiving the at least one external action. Further, the garment may include at least one protective panel 304 attached to at least a part of an inner surface of the crotch portion 300. Further, the at least one protective panel 304 may be configured to extend over the inner surface of the crotch portion 300 for covering the slit 302. Further, the at least one protective panel 304 may be configured to prevent interaction between the zipper and the crotch 502 of the individual based on the covering

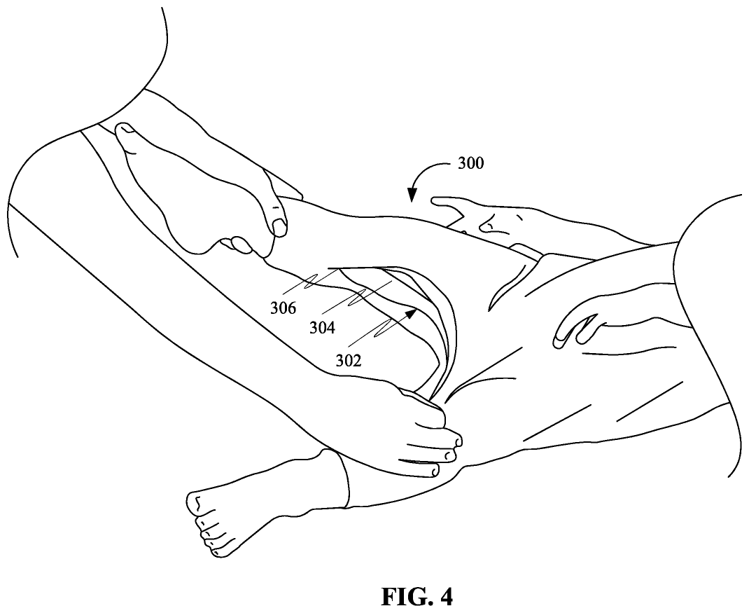

FIG. 4 is a front perspective view of the crotch portion 300, in accordance with some embodiments.

FIG. 5 is a partial front perspective view of the crotch portion 300, in accordance with some embodiments.

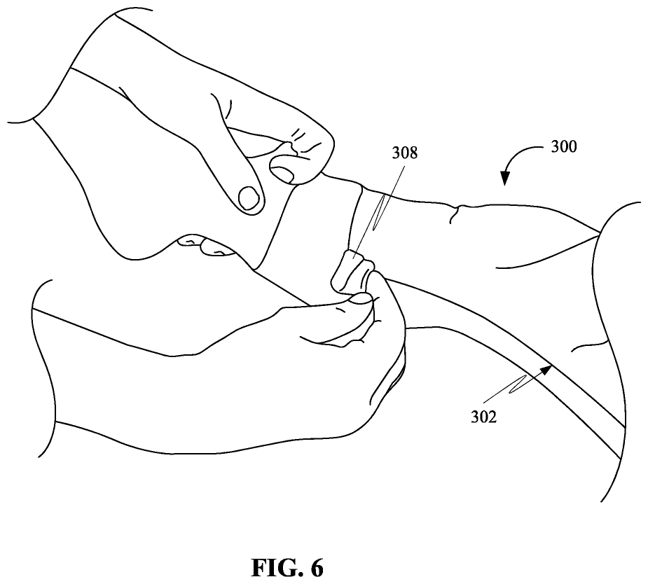

FIG. 6 is a partial front perspective view of the crotch portion 300, in accordance with some embodiments.

FIG. 7 is a partial front perspective view of the crotch portion 300, in accordance with some embodiments.

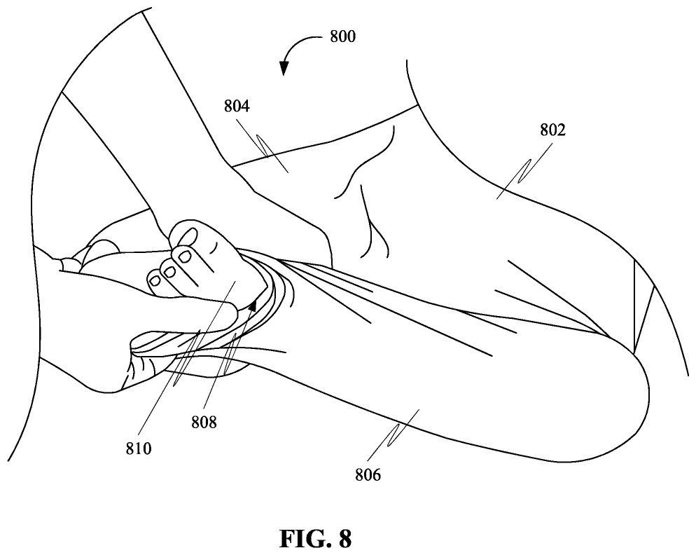

FIG. 8 is a partial perspective view of a garment 800, in accordance with some embodiments. Further, the garment 800 may include a torso portion 802 and two leg portions 804-806. Further, the torso portion 802 may include a neck opening disposed on an upper torso end of the torso portion 802. Further, the torso portion 802 may include two arm openings disposed on two opposing torso side ends of the torso portion 802. Further, each leg portion of the two leg portions 804-806 may include a leg opening 808 disposed on a lower leg end of the each leg portion. Further, the torso portion 802 may be configured to accommodate at least a part of a torso of an individual. Further, the each leg portion may be configured to accommodate at least a part of a leg 810 of the individual. Further, the leg 810 may extend from the leg opening 808 for allowing the each leg portion to accommodate at least the part of the leg 810. Further, the leg 810 may retract through the leg opening 808 for removing the each leg portion from at least the part of the leg 810.

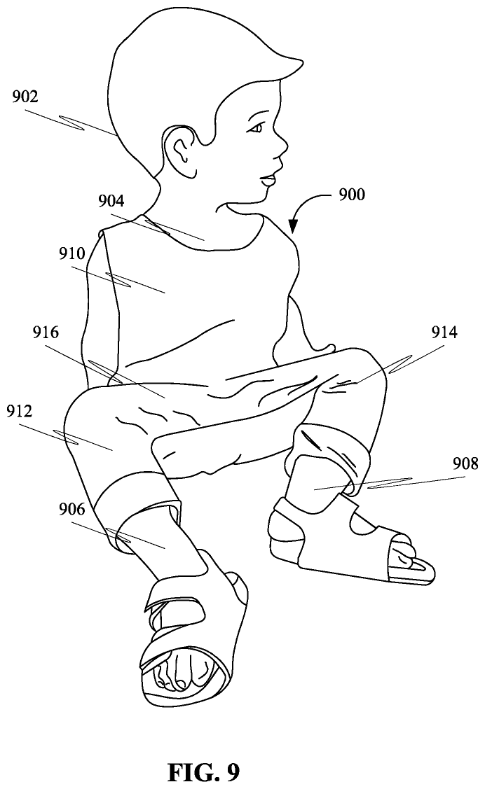

FIG. 9 is a front perspective view of a garment 900 worn by an individual 902, in accordance with some embodiments. Further, the garment 900 may include a torso portion 910 and two leg portions 912-914. Further, the torso portion 910 may include a neck opening disposed on an upper torso end of the torso portion 910. Further, the torso portion 910 may include two arm openings disposed on two opposing torso side ends of the torso portion 910. Further, each leg portion of the two leg portions 912-914 may include a leg opening disposed on a lower leg end of the each leg portion. Further, the torso portion 910 may be configured to accommodate at least a part of a torso 904 of the individual 902. Further, the each leg portion may be configured to accommodate at least a part of a leg 906-908 of the individual 902. Further, a lower torso portion of the torso portion 910 and an upper leg portion of the each leg portion may form a crotch portion 916. Further, the crotch portion 916 may include a slit.

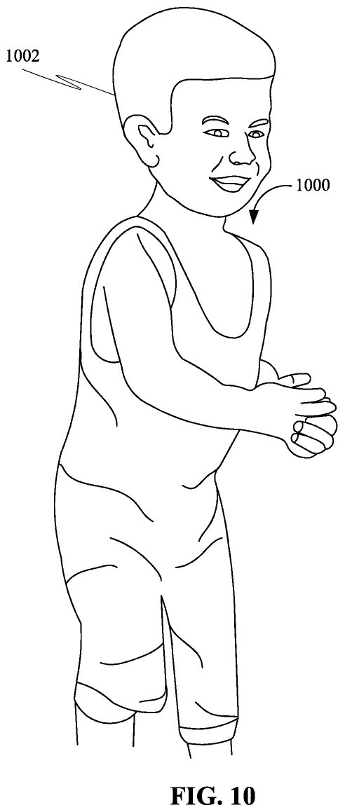

FIG. 10 is a front perspective view of a garment 1000 worn by an individual 1002, in accordance with some embodiments.

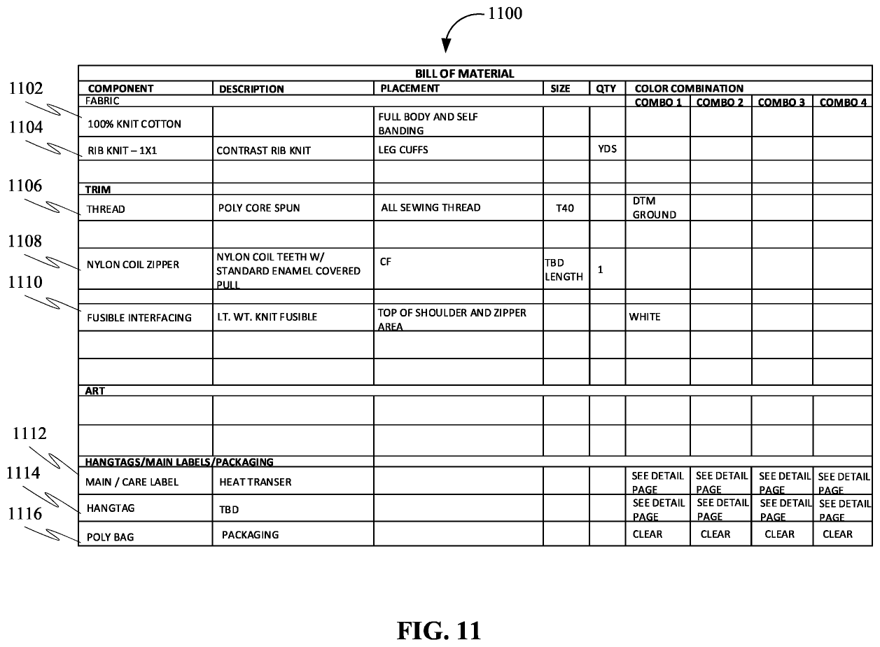

FIG. 11 is a table 1100 of a bill of material associated with a garment, in accordance with some embodiments. Further, a row 1102 of the table 1100 may include 100% knit cotton. Further, the 100% knit cotton may be a fabric component of the bill of material.

Further, the 100% knit cotton may be placed on full body and self banding. Further, a row 1104 of the table 1100 may include a rib knit −1×1. Further, the rib knit −1×1 may be the fabric component of the bill of material. Further, the rib knit −1×1 may include contrast rib knit. Further, the rib knit −1×1 may be placed in leg cuffs. Further, a row 1106 of the table 1100 may include a thread. Further, the thread may be a trim component of the bill of material. Further, the thread may be poly core spun. Further, all sewing thread may include the thread. Further, a size of the thread may be T40. Further, a row 1108 of the table 1100 may include a nylon coil zipper. Further, the nylon coil may be the trim component of the bill of material. Further, the nylon coil zipper may include nylon coil teeth with standard enamel covered pull. Further, a row 1110 of the table 1100 may include fusible interfacing. Further, the fusible interfacing may be the trim component of the bill of material. Further, the fusible interfacing may include light weight knit fusible. Further, a top of a shoulder area and a zipper area may include the fusible interfacing. Further, a row 1112 of the table 1100 may include a main\care label. Further, the main\care label may be a main label component of the bill of material. Further, the main\care label may provide heat transfer. Further, a row 1114 of the table 1100 may include a hangtag. Further, the hangtag may be a hangtag component of the bill of material. Further, a row 1116 of the table 1100 may include a polybag. Further, the polybag may be a packaging component of the bill of material. Further, the polybag may be used for packaging.

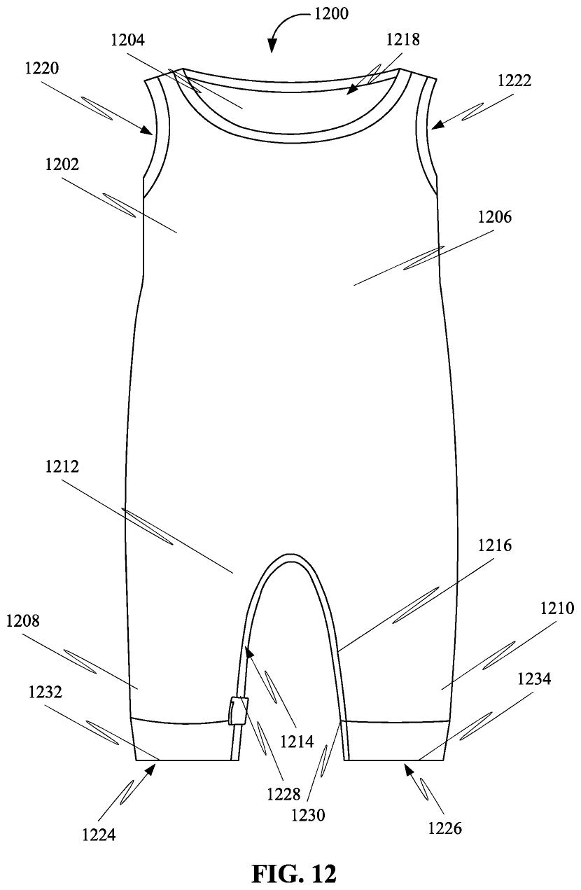

FIG. 12 is a front view of a garment 1200, in accordance with some embodiments. Further, the garment 1200 may include a front panel 1202, a rear panel 1204, and at least one fastener 1216.

Further, the rear panel 1204 may be attached to the front panel 1202 along a periphery of the rear panel 1204 in at least a part forming a torso portion 1206 of the garment 1200 and two leg portions 1208-1210 of the garment 1200. Further, the torso portion 1206 may include a neck opening 1218 disposed on an upper torso end of the torso portion 1206. Further, the torso portion 1206 may include two arm openings 1220-1222 disposed on two opposing torso side ends of the torso portion 1206. Further, each leg portion of the two leg portions 1208-1210 may include a leg opening 1224-1226 disposed on a lower leg end of the each leg portion. Further, the torso portion 1206 may be configured to accommodate at least a part of a torso of an individual. Further, the each leg portion may be configured to accommodate at least a part of a leg of the individual. Further, a lower torso portion of the torso portion 1206 and an upper leg portion of the each leg portion forms a crotch portion 1212. Further, the crotch portion 1212 may include a slit 1214. Further, the slit 1214 may include a first end 1228 and a second end 1230. Further, the each leg portion may include an outer leg portion edge 1232-1234 defining the leg opening 1224-1226. Further, at least one of the first end 1228 and the second end 1230 may be discontiguous with the outer leg portion edge 1232-1234 of the each leg portion. Further, the slit 1214 may be configured to provide access to a crotch of the individual through the slit 1214.

Further, the at least one fastener 1216 may be disposed on the crotch portion 1212. Further, the at least one fastener 1216 may include a first fastener element and a second fastener element. Further, the first fastener element may be disposed on a first crotch portion of the crotch portion 1212 corresponding to a first side of the slit 1214. Further, the second fastener element may be disposed on a second crotch portion of the crotch portion 1212 corresponding to a second side of the slit 1214. Further, the second fastener element and the first fastener element may be configured to be detachably fastened for openably closing the slit 1214. Further, the slit 1214 may transition between an open state and a closed state based on the openably closing. Further, the crotch may be accessible through the slit 1214 in the open state. Further, the crotch may not be accessible through the slit 1214 in the closed state.

In further embodiments, at least one protective panel may be attached to at least a part of an inner surface of the crotch portion 1212. Further, the at least one protective panel may be configured to extend over the inner surface of the crotch portion 1212 for covering the slit 1214. Further, the at least one protective panel may be configured to prevent interaction between the at least one fastener 1216 and the crotch of the individual based on the covering.

Although the present disclosure has been explained in relation to its preferred embodiment, it is to be understood that many other possible modifications and variations can be made without departing from the spirit and scope of the disclosure.

What is claimed is:

1. A garment comprising:
a front panel;
a rear panel attached to the front panel along a periphery of the rear panel in at least a part forming a torso portion of the garment and two leg portions of the garment, wherein the torso portion comprises a neck opening disposed on an upper torso end of the torso portion, wherein the torso portion comprises two arm openings disposed on two opposing torso side ends of the torso portion, wherein each leg portion of the two leg portions comprises a leg opening disposed on a lower leg end of the each leg portion, wherein the torso portion is configured to accommodate at least a part of a torso of an individual, wherein the each leg portion is configured to accommodate at least a part of a leg of the individual, wherein a lower torso portion of the torso portion and an upper leg portion of the each leg portion forms a crotch portion, wherein the crotch portion comprises a slit, wherein the slit comprises a first end and a second end, wherein the slit is configured to provide access to a crotch of the individual through the slit; and
at least one fastener disposed on the crotch portion, wherein the at least one fastener comprises:
a first fastener element disposed on a first crotch portion of the crotch portion corresponding to a first side of the slit; and
a second fastener element disposed on a second crotch portion of the crotch portion corresponding to a second side of the slit, wherein the second fastener element and the first fastener element are configured to be detachably fastened for openably closing the slit, wherein the slit transitions between an open state and a closed state based on the openably closing, wherein the crotch is accessible through the slit in the open state, wherein the crotch is not accessible through the slit in the closed state.

2. The garment of claim 1 further comprising at least one protective panel attached to at least a part of an inner surface of the crotch portion, wherein the at least one protective panel is configured to extend over the inner surface of the crotch portion for covering the slit, wherein the at least one protective panel is configured to prevent interaction between the at least one fastener and the crotch of the individual based on the covering.

3. The garment of claim 1, wherein the at least one fastener comprises a zipper, wherein the first fastener element comprises a first zipper element and the second fastener element comprises a second zipper element, wherein the zipper further comprises a slider operationally coupled with the first zipper element and the second zipper element, wherein the slider is configurable for moving between a plurality of positions along the first zipper element and the second zipper element for detachably fastening the first zipper element and the second zipper element.

4. The garment of claim 3, wherein the first zipper element extends between the first end and the second end on the first crotch portion, wherein the second zipper element extends between the first end and the second end on the second crotch portion, wherein the slider moves between the plurality of positions between the first end and the second end based on at least one external action receivable on at least one portion of the slider, wherein the plurality of positions comprises an open position and a closed position, wherein the slider is positioned proximally to the first end and distally from the second end in the open position, wherein the slider is positioned proximally to the second end and distally from the first end in the closed position.

5. The garment of claim 4 further comprising at least one first panel attached to at least a part of an outer surface of the crotch portion along a periphery of the at least one first panel in at least a part forming a receptacle and an opening leading into the receptacle, wherein the at least one first panel is proximal to at least one of the first end and the second end, wherein the receptacle is configurable for removably receiving the at least one portion of the slider through the opening, wherein receiving of the at least one portion of the slider in the receptacle prevents the at least one portion of the slider form receiving the at least one external action.

6. The garment of claim 1, wherein the each leg portion comprises an outer leg portion edge defining the leg opening, wherein at least one of the first end and the second end is discontiguous with the outer leg portion edge of the each leg portion.

7. The garment of claim 1, wherein the each leg portion comprises an outer leg portion edge defining the leg opening, wherein at least one of the first end and the second end is contiguous with the outer leg portion edge of the each leg portion.

8. The garment of claim 1, wherein the rear panel is detachably attached to the front panel along the periphery of the rear panel in at least the part forming at least one of the torso portion of the garment and the two leg portions of the garment using at least one fastening element.

9. The garment of claim 1, wherein the rear panel is fixedly attached to the front panel along the periphery of the rear panel in at least the part forming the torso portion of the garment and the two leg portions of the garment.

10. The garment of claim 8, wherein the at least one fastening element comprises a primary fastening element and a secondary fastening element, wherein the primary fastening element is disposed along a periphery of the front panel, wherein the secondary fastening element is disposed along the periphery of the rear panel, wherein the primary fastening element is configured to be detachably fastened to the secondary fastening element for detachably attaching the rear panel to the front panel.

11. The garment of claim 1, wherein at least one of the front panel and the rear panel comprises at least one material, wherein the at least one material comprises at least one of at least one stretchable material and at least one flexible material, wherein the at least one material allows at least one of donning and removing of the garment from a body of the individual through at least one of the neck opening and the slit.

12. The garment of claim 11, wherein the at least one material allows the at least one of the donning and the removing of at least one of the two leg portions from the leg through the slit without the at least one of the donning and the removing of the torso portion from the torso.

13. The garment of claim 1 further comprising at least one absorbing layer attached to at least a part of an inner surface of the crotch portion, wherein the at least one absorbing layer comprises at least one absorbing material, wherein the at least one absorbing material is configured for absorbing a liquid disposable on the at least one absorbing layer.

14. The garment of claim 1 further comprising at least one cushioned layer attached to at least a part of an inner surface of the two leg portions, wherein the at least one cushioned layer comprises at least one cushion material, wherein the at least one cushion material is configured for providing cushioning to at least the part of the leg.

15. The garment of claim 1, wherein at least one of the front panel and the rear panel comprises at least one indicator material, wherein the at least one indicator material is configured for reversibly changing coloring of the at least one indicator material between at least one first color and at least one second color in response to an interaction of the at least one indicator material with at least one substance in at least one quantity, wherein the at least one substance is produced by the individual, wherein changing of the coloring from the at least one first color to the at least one second color indicates a presence of the at least one substance, wherein reversing of the coloring from the at least one second color to the at least one first color indicates an absence of the at least one substance.

16. The garment of claim 1, wherein at least one of the front panel and the rear panel comprises at least one first indicator material, wherein the at least one first indicator material is configured for reversibly changing coloring of the at least one first indicator material between at least one first color and at least one second color based on an application of heat in at least one quantity to the at least one indicator material, wherein the heat is associated with a temperature of the individual, wherein changing of the coloring from the at least one first color to the at least one second color indicates an increase in the temperature from at least one first value of the temperature to at least one second value of the temperature, wherein reversing of the coloring from the at least one second color to the at least one first color indicates a decrease in the temperature from the at least one second value to the at least one first value.

17. The garment of claim 1, wherein at least one of the front panel and the rear panel comprises at least one second indicator material, wherein the at least one second indicator material is configured for reversibly changing coloring of the at least one second indicator material between at least one first color and at least one second color based on an application of force in at least one quantity to the at least one second material, wherein changing of the coloring from the at least one first color to the at least one second color indicates an increase in the force from at least one first value of the force to at least one second value of the force, wherein reversing of the coloring from the at least one second color to the at least one first color indicates a decrease in the force from the at least one second value to the at least one first value.

18. The method of claim 1, wherein the torso portion comprises two sleeve portions, wherein a proximal end of each sleeve portion of the two sleeve portions is attached to each arm opening of the two arm openings, wherein the each sleeve portion comprises a sleeve opening disposed on a distal end of the each sleeve portion, wherein the each sleeve portion is configured to accommodate an arm of the individual.

19. A garment comprising:
a front panel;
a rear panel attached to the front panel along a periphery of the rear panel in at least a part forming a torso portion of the garment and two leg portions of the garment, wherein the torso portion comprises a neck opening disposed on an upper torso end of the torso portion, wherein the torso portion comprises two arm openings disposed on two opposing torso side ends of the torso portion, wherein each leg portion of the two leg portions comprises a leg opening disposed on a lower leg end of the each leg portion, wherein the torso portion is configured to accommodate at least a part of a torso of an individual, wherein the each leg portion is configured to accommodate at least a part of a leg of the individual, wherein a lower torso portion of the torso portion and an upper leg portion of the each leg portion forms a crotch portion, wherein the crotch portion comprises a slit, wherein the slit comprises a first end and a second end, wherein the each leg portion comprises an outer leg portion edge defining the leg opening, wherein at least one of the first end and the second end is discontiguous with the outer leg portion edge of the each leg portion, wherein the slit is configured to provide access to a crotch of the individual through the slit; and
at least one fastener disposed on the crotch portion, wherein the at least one fastener comprises:
a first fastener element disposed on a first crotch portion of the crotch portion corresponding to a first side of the slit; and
a second fastener element disposed on a second crotch portion of the crotch portion corresponding to a second side of the slit, wherein the second fastener element and the first fastener element are configured to be detachably fastened for openably closing the slit, wherein the slit transitions between an open state and a closed state based on the openably closing, wherein the crotch is accessible through the slit in the open state, wherein the crotch is not accessible through the slit in the closed state.

20. The garment of claim 19 further comprising at least one protective panel attached to at least a part of an inner surface of the crotch portion, wherein the at least one protective panel is configured to extend over the inner surface of the crotch portion for covering the slit, wherein the at least one protective panel is configured to prevent interaction between the at least one fastener and the crotch of the individual based on the covering.
